(19) United States
(12) Patent Application Publication
(10) Pub. No.: US 2020/0324107 A1
(43) Pub. Date: Oct. 15, 2020

(54) ELECTRODE BODY AND PRODUCTION METHOD FOR ELECTRODE BODY

(71) Applicant: TOHOKU UNIVERSITY, Sendai-shi, Miyagi (JP)

(72) Inventors: Matsuhiko NISHIZAWA, Sendai-shi, Miyagi (JP); Atsuhiro NAKAGAWA, Sendai-shi, Miyagi (JP); Kengo KATO, Sendai-shi, Miyagi (JP); Shotaro YOSHIDA, Sendai-shi, Miyagi (JP)

(73) Assignee: TOHOKU UNIVERSITY, Sendai-shi, Miyagi (JP)

(21) Appl. No.: 16/956,690

(22) PCT Filed: Dec. 25, 2018
(86) PCT No.: PCT/JP2018/047667

§ 371 Date: Jun. 22, 2020

(30) Foreign Application Priority Data
Dec. 22, 2017 (JP) 2017-247039

(51) Int. Cl. A61N 1/04; A61B 5/0408

(52) U.S. Cl. CPC A61N 1/0496; A61B 5/04087

(57) ABSTRACT

This disclosure provides an electrode body in which an electrode is firmly fixed to a gel without performing electrolytic polymerization. In the electrode body, at a least part of a surface of an electrode is uneven, at least a part of the uneven surface of the electrode is covered with a gel, the gel has a three-dimensional network structure, at least a part of the uneven surface is in contact with the three-dimensional network structure and is embedded inside the three-dimensional network structure. The electrode is preferably a laminated body in which a resin layer is laminated on both surfaces of a conductive base material. It is more preferably that the conductive base material be a fabric woven from fibers and the uneven surface be a surface of the fabric.

### TECHNICAL FIELD

This disclosure relates to an electrode body and a method of producing an electrode body.

### BACKGROUND

Measurement of electrical signals generated by a living body such as electrocardiogram, electromyogram and electroencephalogram using various devices, and control of biological functions by electrification (electrostimulation) have been used as common medical diagnosis and treatment methods. In such methods, an electrode body, which is a part of a device, serves as an interface between the device and the living body. Further, in the case of introducing a substance such as a gene by applying an electrical pulse to a tissue (cell), a device including an electrode body may also be used.

The electrode body used in the medical field generally includes conductive wiring such as metal or carbon, and a substrate material (e.g. plastic or glass) having no conductivity. The electrode body to be in direct contact with a living body is required to have biocompatibility, and devices used these days still have room for improvement in this respect.

In recent years, the merit of using a hydrogel, which is excellent in biocompatibility, in a substrate has drawn attention, and technologies of adhering an electrode material to a hydrogel is being developed.

A known example of the adhesion technology is a technology of performing electrolytic polymerization of conductive polymers with a hydrogel placed on an electrode material, and stretching the conductive polymers from the surface of the electrode material in the vicinity of the electrode material to form a conductive adhesive layer (WO 2014/157550 (PTL 1)). In addition, electrodes coated with hydrogel are known technologies (Lee et at al., Nature communications, 2014, 5, 5898 (NPL 1) and Winter et at al., Journal of Biomedical Materials Research Part B, 2006, Volume 81B, Issue 2, May 2007, Pages 551-563 (NPL 2)).

### CITATION LIST

### Patent Literature

PTL 1: WO 2014/157550

### Non-Patent Literature

NPL 1: Lee et at al., Nature communications, 2014, 5, 5898
NPL 2: Winter et at al., Journal of Biomedical Materials Research Part B, 2006, Volume 81B, Issue 2, May 2007, Pages 551-563

### SUMMARY

### Technical Problem

However, in the production method described in PTL 1, the polymers that play a role of adhering the porous body to the electrode are synthesized by electrolytic polymerization, so that it is necessary to provide a conductive polymer layer on the surface layer of the porous body. In addition, since the electrode and the porous body are bound together by the strong entanglement between the conductive polymers and the molecules of the porous body, there has been a demand for technologies of further firmly fixing the electrode to the porous body to make the bond more stable.

It could thus be helpful to provide an electrode body in which an electrode is firmly fixed to a gel without performing electrolytic polymerization.

### Solution to Problem

The primary features of the present disclosure are as follows.

In the electrode body of the present disclosure, at a least part of a surface of an electrode is uneven, at least a part of the uneven surface of the electrode is covered with a gel, the gel has a three-dimensional network structure, and at least a part of the uneven surface is in contact with the three-dimensional network structure and is embedded inside the three-dimensional network structure.

It is preferable that the thickness of the electrode be 1 μm to 500 μm.

It is preferable that the electrode be a laminated body in which a resin layer is laminated on both surfaces of a conductive base material.

It is preferable that the conductive base material be a fabric woven from fibers and the uneven surface be a surface of the fabric. It is more preferable that the conductive base material be carbon fabric.

It is preferable that an exposed portion where the conductive base material is exposed be provided on at least a part of the surface of the electrode. It is preferable that the exposed portion be in contact with the three-dimensional network structure and be embedded inside the three-dimensional network structure.

It is preferable that the gel be a hydrogel.

It is preferable that the gel contain polyvinyl alcohol.

It is preferable that the electrode be curved at at least one location, and the curve be in contact with the three-dimensional network structure and be embedded inside the three-dimensional network structure.

It is preferable to include two or more of the electrodes.

The method of producing an electrode body of the present disclosure includes an electrode forming step of forming an electrode, an immersion step of immersing the electrode in a gel-forming solution, and a gelling step of making the gel-forming solution into a gel with the electrode immersed in the gel-forming solution.

It is preferable to repeat freezing and thawing twice or more in the gelling step.

It is preferable that at least a part of a surface of the electrode be uneven, at least a part of the uneven surface be covered with a gel, the gel have a three-dimensional network structure, and at least a part of the uneven surface be in contact with the three-dimensional network structure and be embedded inside the three-dimensional network structure.

It is preferable that the thickness of the electrode be 1 μm to 500 μm.

It is preferable that the electrode be a laminated body in which a resin layer is laminated on both surfaces of a conductive base material.

It is preferable that the conductive base material be carbon fabric.

It is preferable that an exposed portion where the conductive base material is exposed be provided on at least a part of the surface of the electrode.

It is more preferable that the gel be a hydrogel.

It is preferable that the gel contain polyvinyl alcohol.

### Advantageous Effect

Because the electrode body of the present disclosure has the structure described above, the electrode is firmly fixed to the gel.

### BRIEF DESCRIPTION OF THE DRAWINGS

In the accompanying drawings:

FIG. 1 schematically illustrates an example of the electrode body of the present embodiment (which is a perspective view);

FIG. 2 schematically illustrates an example of the electrode body of the present embodiment (which is a cross-sectional view taken along the line X-X in FIG. 1);

FIG. 3 schematically illustrates an example of the electrode in the electrode body of the present embodiment;

FIGS. 4A and 4B schematically illustrate examples of the curve of the electrode (which are cross-sectional views);

FIGS. 5A to 5E schematically illustrate an example of the electrode forming step;

FIGS. 6A to 6D schematically illustrate an example of the electrode forming step;

FIGS. 7A to 7D schematically illustrate an example of the immersion step;

FIGS. 8A to 8D schematically illustrate an example of the immersion step;

FIG. 9 schematically illustrates the mold used in the immersion step of Example 1 (which is a plan view);

FIG. 10 schematically illustrates the electrode used in Example 1 (which is a cross-sectional view);

FIG. 11 schematically illustrates the electrode body obtained in Example 1;

FIG. 12 is a photograph of the electrode body obtained in Example 1;

FIGS. 13A and 13B schematically illustrate the molds used in the immersion step of Example 2 (which are plan views);

FIG. 14 schematically illustrates the electrode body obtained in Example 2 (which is a cross-sectional view);

FIG. 15 schematically illustrates the dimensions of the electrode used in Example 2, where the upper is a plan view, and the lower is a cross-sectional view taken along the line X-X of the plan view;

FIGS. 16A and 16B schematically illustrate the dimensions of the electrode body obtained in Example 2, where FIG. 16A is a cross-sectional view taken along the line Y-Y of FIG. 16B, and FIG. 16B is a plan view;

FIGS. 17A to 17C are photographs of the electrode body obtained in Example 2, where FIG. 17A is a photograph taken from the side, FIG. 17B is a photograph taken from the back (electrode exposed portion), and FIG. 17C is a photograph taken from the front (taken-out wiring portion);

FIG. 18 illustrates the measurement point of the sheet resistance measured in Example 2, from the tip of the wiring of the electrode to the center of the exposed portion;

FIGS. 19A and 19B illustrate embedding an electrode body behind the neck of a rat, where FIG. 19A is a photograph of the state of being embedded in a rat, and FIG. 19B is a photograph of the wiring taken out from the back of the neck after the operation;

FIGS. 20A and 20B are photographs illustrating the results of the followability to gel, where FIG. 20A is a photograph taken from the above of the samples, and FIG. 20B is a photograph taken from the side;

FIG. 21 illustrates the results of the electric double layer capacity;

FIG. 22 is an analysis diagram of the AC impedance spectrum;

FIGS. 23A and 23B are photographs of evaluation of combination of the gel and the electrode, where FIG. 23A is the result of the sample (CF), and FIG. 23B is the result of the sample (plastic film);

FIGS. 24A and 24B illustrate a method of evaluating mechanical strength and the evaluation results, where FIG. 24A explains the evaluation method, and FIG. 24B illustrates the evaluation results;

FIG. 25 schematically illustrates the electrode body obtained in Example 3;

FIG. 26 are photographs in which an electrode is fixed to the brain of a rat for electroencephalogram measurement; and

FIG. 27 illustrates the results of the electroencephalogram measurement.

### DETAILED DESCRIPTION

Hereinafter, embodiments of the electrode body and the method of producing an electrode body of the present disclosure will be described in detail with reference to the drawings.

[Electrode Body]

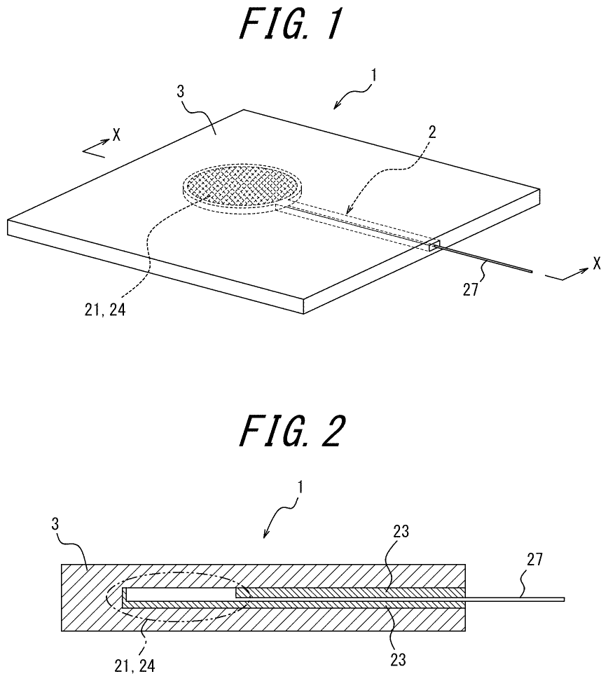

FIG. 1 is a perspective view of an electrode body of an example of the present embodiment, and FIG. 2 is a cross-sectional view taken along the line X-X in FIG. 1.

As illustrated in FIGS. 1 and 2, in the electrode body 1 of the present embodiment, at least a part of a surface of an electrode 2 is uneven, and at least a part of the uneven surface 21 is covered with a gel 3. In the electrode 2, the uneven surface 21 is an exposed portion 24, and portions excluding the uneven surface 21 are covered with a resin layer 23. The exposed portion 24 and the resin layer 23 of the electrode 2 are covered with the gel 3.

The gel 3 has a three-dimensional network structure, and the exposed portion 24 has an uneven surface that is in contact with the three-dimensional network structure and is embedded inside the three-dimensional network structure. The resin layer 23 also is in contact with the three-dimensional network structure of the gel 3 and is embedded inside the three-dimensional network structure. Because the exposed portion 24 has an uneven surface, the three-dimensional network structure of the gel enters the irregular grooves, which firmly fixes the electrode to the gel by the anchor effect.

When the electrode has a plurality of through holes penetrating in the thickness direction, the three-dimensional network structure of the gel may enter the through holes, and the network of the three-dimensional network structure may pass through the inside of the electrode and penetrate in the thickness direction of the electrode. In this case, the electrode is fixed in the thickness direction by the three-dimensional network structure of the gel, so that the electrode can be further firmly fixed to the gel.

In the case of an electrode having a network structure on the surface and inside the electrode such as a fabric, the network structure of the conductive base material and the three-dimensional network structure of the gel are entangled with each other, and the electrode is fixed in all directions such as thickness and width. As a result, the electrode can be further firmly fixed to the gel.

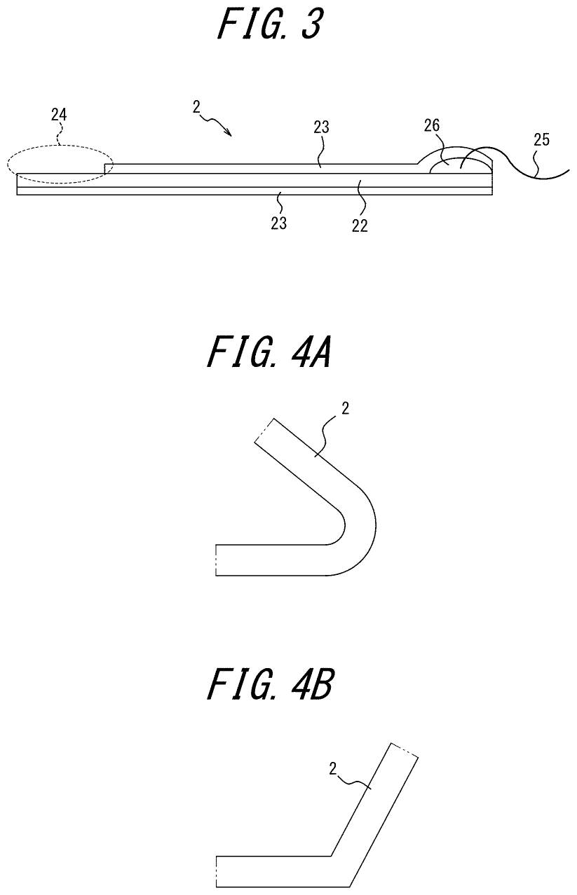

The electrode 2 may have one end covered with the gel 3 and the other end not covered with the gel 3. The one end covered with the gel 3 of the electrode 2 may include, for example, the exposed portion 24 having an uneven surface. The other end of the electrode 2 that is not covered with the gel may be a lead connecting portion 27 (FIGS. 1 and 2) or may be electrically connected to wiring 25 through a connecting portion 26 (FIG. 3).

In the electrode body 1 of the present embodiment, the uneven surface 21 of the electrode 2 is covered with the gel 3, and the uneven surface 21 is in contact with the three-dimensional network structure of the gel and is embedded inside the three-dimensional network structure. Therefore, the electrode 2 hardly slips off or comes off in the gel 3 and is excellent in fixability. In particular, because high molecular weight polymers starting from the surface of the electrode and extending into the gel (porous body) are tightly entangled with the molecules of the gel, the electrode and the gel are less likely to slip off during use and the fixation can be maintained for a longer period of time than in the case of adhering the gel to the electrode.

In addition, this does not require an adhesion step of polymerizing high molecular weight polymers, which facilitates the production.

Further, this does not cause deterioration of interfacial electrical properties due to the bond of high molecular weight polymers in the electrode surface.

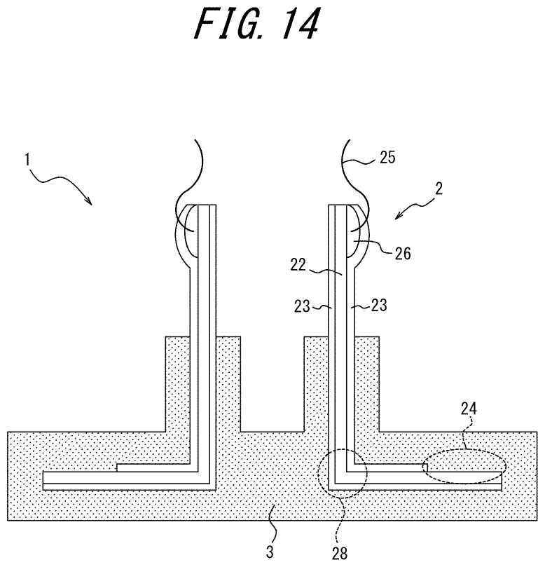

Particularly when the uneven surface 21 is a surface of a fabric, the gel 3 (particularly the three-dimensional network structure of the gel 3) penetrates into the fabric, and the gel 3 and the fabric are intricately entwined with each other. As a result, the fabric can be further firmly fixed to the gel 3. In addition, when the electrode 2 is curved at one or more locations and the curve 28 is covered with the gel 3 (see FIG. 14), the electrode 2 is further firmly fixed to the gel 3. It is preferable that the curve 28 be in contact with the three-dimensional network structure and be embedded inside the three-dimensional network structure.

The above example describes the case where the exposed portion 24 is uneven. However, the resin layer 23 may be an uneven portion, or the resin layer 23 may be fibers or other materials that can be easily penetrated by the gel.

(Electrode)

Examples of the electrode include a conductive base material such as a carbon electrode, a metal electrode, a stretchable electrode, and a composite electrode thereof. Examples of the composite electrode include a combination of a metal electrode and a carbon electrode, a combination of a metal electrode and a stretchable electrode, and a combination of a carbon electrode and a stretchable electrode. For example, it is possible to use a composite electrode where carbon particles, which are carbon electrodes, are embedded in the surface of a stretchable electrode. Among these, a carbon electrode is preferable from the viewpoint of flexibility and long-term stability of the resistance value of the electrode.

The electrode 2 may be a body of single layer of the conductive base material 22 described above or may be a laminated body including, for example, the conductive base material 22 and a resin layer 23 such as an insulating resin layer. For example, it may be a laminated body where the resin layer 23 is laminated on both surfaces of the conductive base material 22 (FIGS. 2 and 3), or a laminated body where the resin layer 23 is laminated on one surface of the conductive base material 22. Among these, it is preferable to have an exposed portion 24 where the conductive base material 22 is exposed in at least a part of the surface of the electrode 2 (FIGS. 2 and 3), from the viewpoint that electricity can be supplied partially by covering the conductive base material with an insulating resin layer and exposing only a part of the surface of the conductive base material.

The electrode 2, as a connection structure that connects to an external power source or the like, may include wiring 25 and a connecting portion 26 for electrically connecting the wiring 25 to the conductive base material 22 (FIG. 3), or may be directly connected to an external power source or the like via a lead connecting portion 27 of the conductive base material 2 (FIG. 2). The connecting portion 26 is preferably provided on the surface of the conductive base material 22 on the same side as the exposed portion 24. Further, it is preferable that the connecting portion 26 and/or the lead connecting portion 27 be provided on the opposite side of the exposed portion 24 with the curve 28 of the electrode interposed therebetween.

Specific examples thereof include an electrode 2 that is a laminated body where a resin layer 23 (e.g. an insulating resin layer such as PDMS) is provided on both surfaces of a conductive base material 22 (e.g. carbon fabric), an exposed portion 24 where one surface of the conductive base material 22 is exposed is provided near one end, and a lead connecting portion 27 where both surfaces are exposed is provided near the other end, as illustrated in FIG. 2; and an electrode 2 that is a laminated body where a resin layer 23 (e.g. an insulating resin layer such as PDMS) is provided on both surfaces of a conductive base material 22 (e.g. carbon fabric), an exposed portion 24 where one surface of the conductive base material 22 is exposed is provided near one end, and wiring 25 is provided via a connecting portion 26 near the other end, as illustrated in FIG. 3.

—Carbon Electrode—

The carbon electrode is not particularly limited as long as it can be used as an electrode. Specific examples thereof include a graphene sheet, an aggregate of carbon nanotubes, an aggregate of carbon particles, and a carbon cloth such as carbon fabric. Examples of the carbon fabric include a fabric woven from fibers impregnated with carbon nanotubes. Among these, carbon fabric is preferable from the viewpoint of being excellent in flexibility and long-term stability of the resistance value of the electrode.

It is also acceptable to use a fabric impregnated with a conductive material such as conductive polymers or metal particles, yet carbon fabric is preferable from the viewpoint of biocompatibility.

—Metal Electrode—

The metal electrode is not particularly limited as long as it can be used as an electrode. Specific examples thereof include gold, platinum, titanium, aluminum, and tungsten. Among these, gold or platinum is preferable from the viewpoint of stability and excellent biosafety.

—Stretchable Electrode—

The stretchable electrode is not particularly limited as long as it is an elastomer that can be used as an electrode. That is, it is possible to use an elastomer having ionic conductivity or conductivity. Specific examples include polyurethane, silicone rubber, and fluororubber, among which polyurethane is preferred.

—Resin Layer—

The resin layer 23 may be laminated so as to be in contact with the conductive base material 22, or may be laminated via another layer. Among these, it is preferable that the resin layer 23 be in contact with the conductive base material 22 from the viewpoint of weight reduction.

Examples of the material forming the resin layer 23 include polydimethylsiloxane, polyurethane, polypropylene, polylactic acid, poly(lactide-co-glycolide) copolymer, polydioxanone, acrylonitrile butadiene styrene copolymer, acrylic ester, acrylonitrile ethylene propylene rubber styrene copolymer, acrylonitrile styrene copolymer, acrylonitrile styrene acrylate, polybutadiene, bismaleimide triazine, cellulose acetate, cellulose acetate butyrate, cellulose acetate propionate, cyclic butyl terephthalate, cresol formaldehyde, carboxymethyl cellulose, nitrocellulose, hydrin rubber, cellulose propionate, chlorinated vinyl chloride, chloroprene rubber, casein, cellulose triacetate, diallyl phthalate, ethylene chlorotrifluoroethylene copolymer, ethylenediaminetetraacetic acid, ethylene ethyl acrylate, ethylene methyl acrylate, ethylene methacrylic acid, epoxy resin, ethylene propylene diene terpolymer, ethylene tetrafluoroethylene copolymer, ethylene vinyl acetate copolymer, ethyl vinyl ether, perfluoro rubber, polyethylene, polystyrene, butyl rubber, isoprene rubber, diphenylmethane isocyanate, melamine formaldehyde, nitrile rubber, polymethyl methacrylate, polyimide, polyethylene terephthalate, polycarbonate, polyetheretherketone, polyisobutylene, polymethylmethacrylate, polyvinyl acetate, polyvinyl chloride, nylon, polyvinylidene fluoride, polyvinyl alcohol, polyvinyl pyrrolidone, styrene butadiene, silicone, polyester, Teflon® (Teflon is a registered trademark in Japan, other countries, or both), and polytetrafluoroethylene. Among these, an insulating resin is preferable, and polydimethylsiloxane (PDMS) is more preferable from the viewpoint of fixability to the conductive base material.

By providing a resin layer as describe above, it is easy to maintain the curved structure of the conductive base material even in the case of using a conductive base material with high flexibility.

—Exposed Portion—

The exposed portion 24 may be provided at one location or at a plurality of locations on the surface of the electrode. It is preferable that at least a part of the exposed portion 24 be provided with the uneven surface, and it is more preferable that the whole exposed portion 24 be provided with the uneven surface.

The exposed portion 24 may be provided on both surfaces of the electrode, or may be provided on one surface. Among the above, the exposed portion 24 is preferably provided on one surface from the viewpoint of being able to supply electricity partially. In the case where a conductive base material not covered with a resin layer is used as the electrode, the entire surface of the electrode is the exposed portion.

—Wiring—

Common wiring can be used as the wiring 25.

—Connecting Portion—

The connecting portion 26 is preferably made of the same material as the material forming the conductive base material from the viewpoint of conductive efficiency. For example, when carbon fabric is used as the conductive base material 22, the connecting portion 26 is preferably formed by drying a dispersion of carbon nanotubes.

—Lead Connecting Portion—

Examples of the lead connecting portion 27 include one end of the conductive base material 22 not covered with the gel 3 (e.g. the end opposite to the exposed portion) (FIG. 2).

—Curve—

The electrode 2 preferably has at least one curve 28 from the viewpoint of further firmly fixing the electrode to the gel.

The curve 28 may be in a curved shape (FIG. 4A), a bent shape (FIG. 4B), a twisted shape, or the like. Among these, a curved shape (FIG. 4A) is preferable from the viewpoint of easy production.

In the electrode 2, the number of the curve 28 is preferably at least one and may be more than one. When there is a plurality of curves 28, each curve may be provided continuously or may be provided at intervals.

The radius of curvature of the curve 28 is preferably more than 0 mm from the viewpoint of the fixability between the electrode and the gel. For example, when an exposed portion 24 is provided, the center of curvature of the curve may be on the exposed portion 24 side.

When a plurality of curves 28 are provided, the centers of curvature of the respective curves may be on the same side or on different sides. Further, the radii of curvature of the respective curves may be the same or different.

The radius of curvature of the curve may be a radius of curvature at any one point within the curve in a cross section in the thickness direction of the electrode. As used herein, the cross section in the thickness direction of the electrode is a cross section cut in the laminating direction of the conductive base material and the resin layer, which is a cross section including the curve.

The thickness of the electrode 2 is preferably 1 μm to 500 μm and more preferably 1 μm to 300 μm from the viewpoint of flexibility of the electrode body.

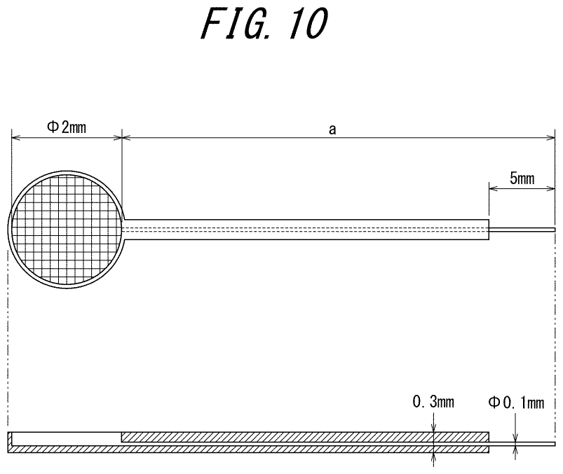

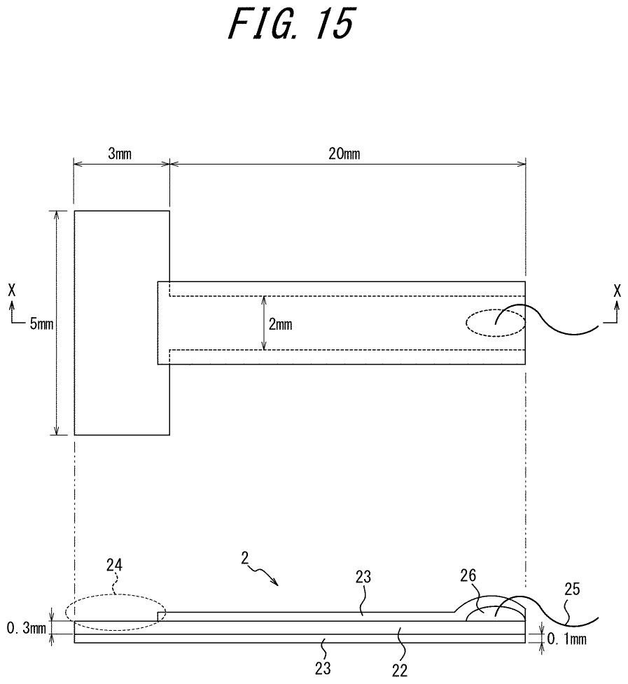

The shape of the electrode 2 in a plan view before being curved is not particularly limited. For example, it may be in a substantially polygonal shape such as a rectangle, a substantially circular shape, or a ladle shape (FIG. 10), or may be in a T-shape (FIG. 15), a triangular shape or the like where, in the length direction, one end is narrow and the other end is wide. The curve is preferably provided between the narrow end and the wide end.

The shape of the electrode 2 after being curved is not particularly limited. For example, it may have a shape where a curve is provided on any substantially straight line on the electrode surface. In addition, the exposed portion may be deformed so as to conform to the shape of an animal, an organ, etc. to which it is applied.

At least a part of the surface of the electrode 2 is uneven.

Examples of the unevenness include saw-toothed unevenness in a cross section in the thickness direction of the electrode, unevenness having a plurality of through holes penetrating in the thickness direction of the electrode, and surface unevenness formed by a fabric woven from fibers. Among these, unevenness having a plurality of through holes and surface unevenness formed by a fabric is preferable from the viewpoint of further firmly fixing the electrode to the gel, and surface unevenness formed by a fabric is more preferable from the viewpoint of excellent followability to the gel and excellent flexibility.

Examples of the fabric forming the surface unevenness include a carbon cloth such as the carbon fabric described above, and a fabric impregnated with a conductive material. Carbon fabric is preferable from the viewpoint of biocompatibility.

The uneven surface 21 may be provided on any part of the electrode 2, yet it is preferably provided on the exposed portion 24 from the viewpoint of preventing a measurement site from slipping off.

(Gel)

The gel 3 is preferably one having excellent flexibility and biocompatibility and is more preferably a hydrogel. The gel 3 is preferably an ionic conductor. The gel preferably has a three-dimensional network structure. Examples of the three-dimensional network structure (gel network) include a structure having linear portions and coupling portions that couple the linear portions.

Hydrogel is a gel in which water is retained as a solvent in a three-dimensional network structure, which exhibits extremely excellent water absorption. Gels, whether natural or synthetic, are often hydrogels that contain water. Most of the soft tissues that make up a living body, such as cornea, crystalline lens, vitreous body, muscles, blood vessels, axons, and cartilage, are typical hydrogels containing 60% to 80% water in the network structure of biopolymers. Further, although hard tissues such as bones and teeth are not hydrogels themselves, they often have a structure in which a gel-like substance such as collagen is filled in gaps of inorganic hydroxyapatite. Therefore, there are many hydrogels that are derived from a living body and are excellent in biocompatibility.

Specific examples of the hydrogel include agarose gel, collagen gel, glucomannan gel, polyacrylamide gel, polyacrylamide-2-methylpropanesulfonic acid gel, fibrin gel, polyvinyl alcohol gel, polyhydroxyethyl methacrylate gel, silicone hydrogel, polyvinylpyrrolidone gel, polyethylene glycol gel, poly-2-acrylamide-2-methylpropanesulfonic acid gel, alginic acid gel, carrageenan gel, chitosan gel, poly-N-isopropylacrylamide gel, acrylic acid gel, polystyrene sulfonate gel, and a mixture of two or more of these (composite gel). Among these, a hydrogel containing polyvinyl alcohol is preferable, and a hydrogel consisting only of polyvinyl alcohol is more preferable, from the viewpoint of having high safety to living bodies and having no biodegradability.

The gel can include materials other than the material forming the gel as long as the effects of the present disclosure can be obtained. Specific examples include cells, proteins (antibodies, antigens, enzymes, cell growth factors, etc.), nucleic acids such as DNA and RNA, peptide molecules, microparticles and nanoparticles, fluorescent and phosphorescent molecules, and redox agents.

The water content of the gel is not particularly limited. However, it is preferably 60 mass % to 99.5 mass %, more preferably 70 mass % to 99 mass %, and further preferably 80 mass % to 99 mass %.

In the electrode body of the present embodiment, the gel may cover the entire electrode (for example, the entire electrode except the connection structure such as the wiring 25 and the lead connecting portion 27) (FIGS. 2 and 3), or the gel may cover a part of the electrode such as the uneven surface 21 and the curve 28.

In the electrode body of the present embodiment, the gel covers at least a part of the uneven surface 21 of the electrode. The gel may cover all over the uneven surface 21 or may cover a part of the uneven surface 21. In the case where there is a plurality of uneven surfaces 21, it is preferable that the gel cover at least one uneven surface 21, and the gel may cover all the uneven surfaces 21.

Further, it is preferable that the gel cover the exposed portion 24 and/or the curve 28 of the electrode. In the case where there is a plurality of exposed portions 24 and/or curves 28, it is preferable that the gel cover at least one exposed portion 24 and/or curve 28, and the gel may cover all the exposed portions 24 and/or curves 28.

The electrode 2 may be covered with the gel 3 by being in contact with the gel 3 or may be covered with the gel 3 via another layer. Among these, it is preferable that the electrode 2 be in contact with the gel 3 from the viewpoint of further firm fixing.

Note that the “cover” means that the entire surface of the target region of the electrode 2 is covered with the gel 3.

The “cover” preferably means that the entire surface of the target region is in contact with the three-dimensional network structure of the gel and is embedded inside the three-dimensional network structure.

For the electrode body 1 of the present embodiment, the number of the electrode 2 may be one or two or more (for example, 2 to 64). For example, in the case where the device is used for electrical stimulation of muscles, the number of the electrode may be two, and in the case where the device is used not only for electrical stimulation but also for sensing brain waves, the number may be 2 to 64 from the viewpoint of obtaining more detailed information.

For the electrode body of the present embodiment, the combination of the electrode 2 and the gel 3 is preferably, for example, a combination of an electrode that is a laminated body of carbon fabric and PDMS, and a hydrogel that contains polyvinyl alcohol, from the viewpoint of long-term stability of the resistance value of the electrode, difficulty in peeling off the gel from the electrode, and safety in vivo.

The electrode body of the present embodiment can be used as a gel electrode that is embedded in a living body by, for example, being attached to an organ of a living body, or being embedded under the skin. Specifically, it can be used as a measurement stimulating electrode to be attached to an organ, an electrode for stimulating a throat muscle, an electrode to be attached to the brain surface, or an electrode to be inserted into a gap in the brain, for example.

The electrode body of the present embodiment may be embedded so that the portions covered with the gel are embedded inside a body and the portions not covered with the gel are exposed outside the body.

[Method of Producing Electrode Body]

The method of producing an electrode body of the present embodiment may be a method including, for example, an electrode forming step of forming an electrode, an immersion step of immersing the electrode in a gel-forming solution, and a gelling step of making the gel-forming solution into a gel with the electrode immersed in the gel-forming solution.

(Electrode Forming Step)

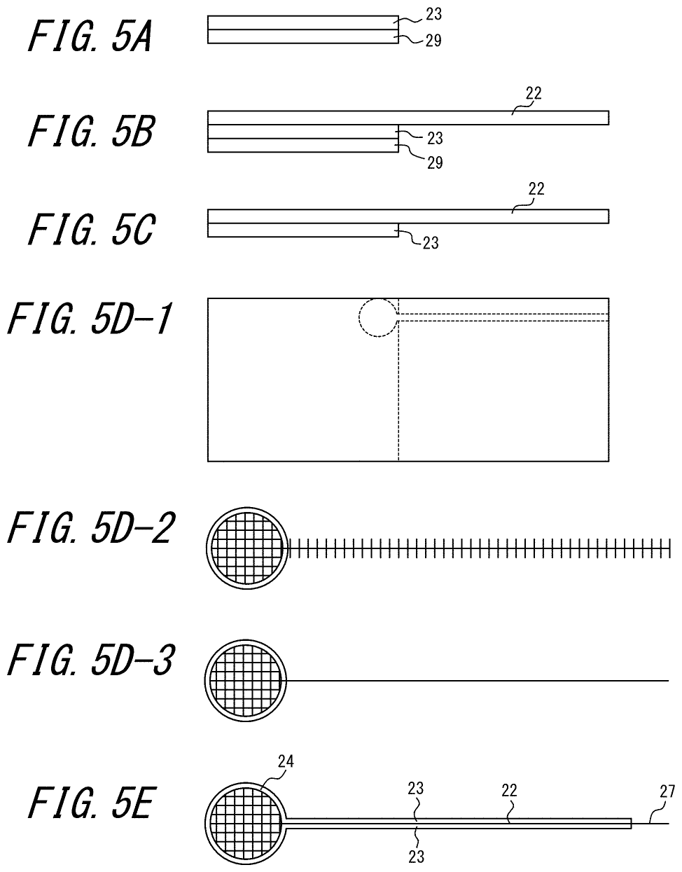

FIGS. 5A to 5E schematically illustrate an example of the electrode forming step.

In the electrode forming step, a resin layer 23 is formed on a substrate 29 for forming electrode (FIG. 5A). The formed resin layer 23 is further applied with a material forming the resin layer, and a conductive base material 22 larger than the resin layer 23 is placed thereon and cured (FIG. 5B). Subsequently, the laminated body of the conductive base material 22 and the resin layer 23 is peeled off from the substrate 29 for forming electrode (FIG. 5C) and cut into a predetermined shape (FIG. 5D-1)). For example, when the conductive base material 22 is carbon fabric, fibers fray in portions where the resin layer 23 is not laminated (FIG. 5D-2), so that it is acceptable to keep one fiber and remove other fibers (FIG. 5D-3). It is covered with the material forming the resin layer except for an exposed portion 24 on one end side and a lead connecting portion 27 on the other end side, and the material is cured (FIG. 5E). In this way, an electrode 2 in which an exposed portion 24 is provided on one end side, a lead connecting portion 27 is provided on the other end side, and a resin layer 23 is laminated on both surfaces of a conductive base material 22 is obtained.

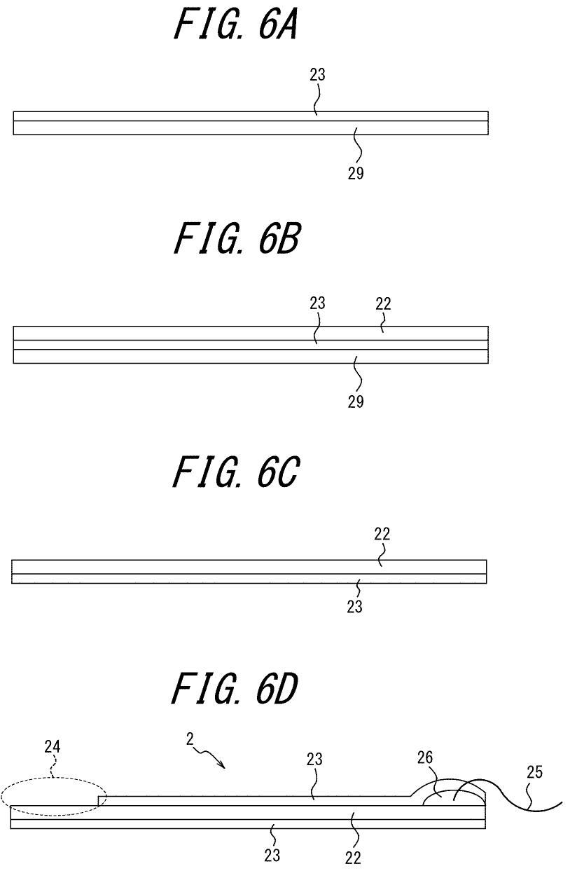

FIGS. 6A to 6D schematically illustrate another example of the electrode forming step.

In the electrode forming step, a resin layer 23 is formed on a substrate 29 for forming electrode (FIG. 6A). The formed resin layer 23 is further applied with a material forming the resin layer, and a conductive base material 22 is placed thereon and cured (FIG. 6B). Subsequently, the laminated body of the conductive base material 22 and the resin layer 23 is peeled off from the substrate 29 for forming electrode (FIG. 6C). A connecting portion 26 for connecting to wiring 25 is provided on one end side of the conductive base material 22, the conductive base material 22 is covered with the material forming the resin layer except for an exposed portion 24, and the material is cured (FIG. 6D). In this way, an electrode 2 in which a connecting portion 26 for connecting to wiring 25 is provided at one end, an exposed portion 24 is provided on the other end, and a resin layer 23 is laminated on both surfaces of a conductive base material 22 is obtained.

Examples of the substrate 29 for forming electrode include a plate made of glass, plastic, cloth, wood, or the like. Among these, a glass plate is preferable because it is flat and has low adhesion to the electrode.

Examples of the method of applying the material forming the resin layer onto the substrate 29 for forming electrode include spin coating and spray coating.

The conditions of the application can be appropriately determined depending on the viscosity of the material to be applied, the thickness of the layer to be formed, and the like. For example, the conditions for spin coating PDMS on a slide glass may be a rotation rate of 1000 rpm to 2000 rpm and a time of 20 seconds to 60 seconds.

The method of curing the material forming the resin layer can be appropriately determined depending on the material used. For example, a method of placing the substrate for forming electrode on a hot plate having a temperature of 120° C. or higher and heating the substrate may be used.

Examples of the method of applying the material forming the resin layer onto the resin layer include spin coating and spray coating. For example, a method of applying PDMS on a PDMS layer with a rotation rate of 500 rpm to 600 rpm and a time of 20 seconds to 30 seconds may be used. The rotation rate is preferably lower than that when forming the resin layer on the substrate for forming electrode, from the viewpoint of uniformly applying the resin without gap, obtaining a resin layer having an appropriate thickness, and further firmly adhering the resin layer to the electrode.

Examples of the method of providing the connecting portion 26 include a method of placing the wiring 25 on the conductive base material 22, dropping a few drops of carbon nanotube dispersion or the like, and drying it.

Examples of the method of providing the exposed portion 24 include a method of covering portions other than a region to be exposed on the conductive base material 22 with the material forming the resin layer, and, for example, heating it to cure the material.

(Immersion Step)

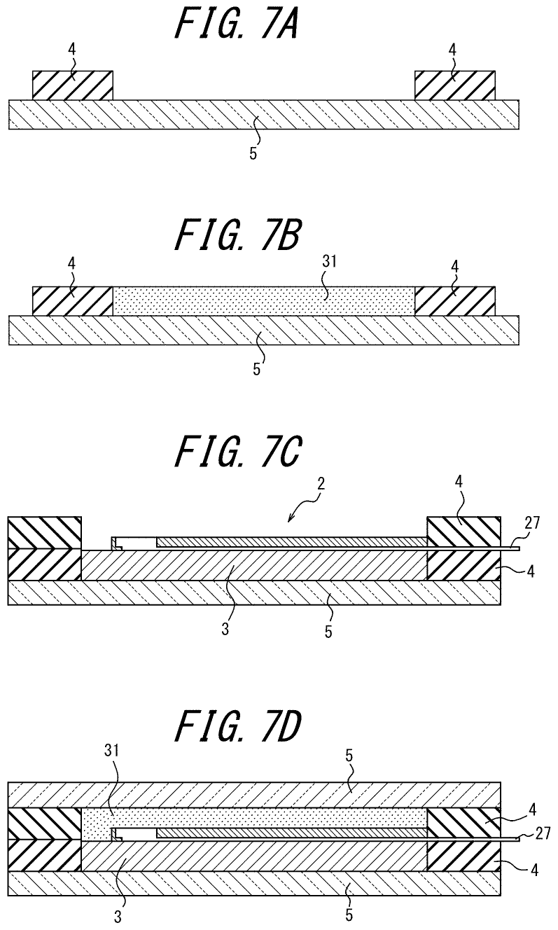

FIGS. 7A to 7D schematically illustrate an example of the immersion step.

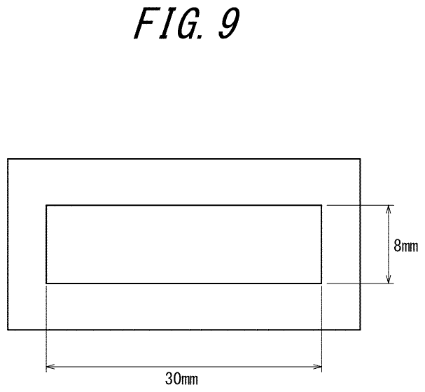

An electrode body mold 4 having a through hole in the center is placed on a substrate 5 for forming electrode body (FIGS. 7A and 9). A gel-forming solution 31 is poured into the through hole of the electrode body mold 4 (FIG. 7B), and the gel-forming solution is frozen and thawed to make the gel-forming solution into a gel. An electrode body mold 4 of the same shape is stacked with the lead connecting portion 27 on the frame of the electrode body mold 4 (FIG. 7C), and the gel-forming solution 31 is poured into the mold and pressed from above by a substrate 5 for forming electrode body (FIG. 7D).

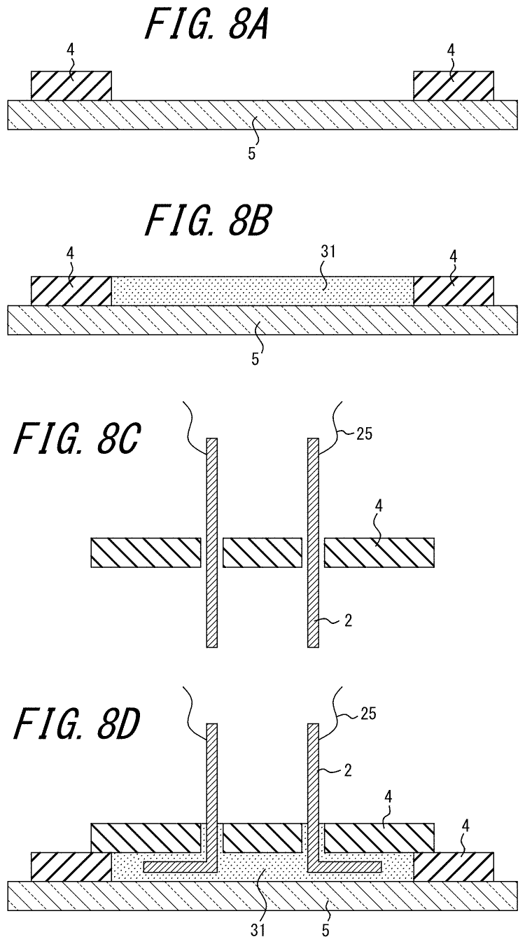

FIGS. 8A to 8D schematically illustrate another example of the immersion step.

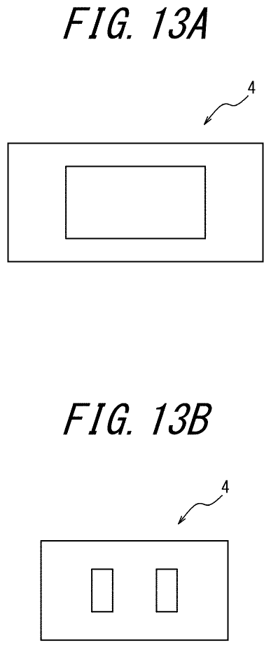

An electrode body mold 4 having a through hole in the center is placed on a substrate 5 for forming electrode body (FIGS. 8A and 13A), and a gel-forming solution 31 is poured into the through hole of the electrode body mold 4 (FIG. 8B). The electrodes 2 described above are inserted into another electrode body mold 4 that is provided with through holes through which the electrode can be inserted (FIGS. 8C and 13B), and the other electrode body mold 4 into which the electrodes are inserted is pressed to the electrode body mold 4 filled with the gel-forming solution 31 from above (FIG. 8D).

Examples of the electrode body mold 4 include a mold made of silicone rubber, PDMS, or the like.

The shape of the mold is not particularly limited as long as an electrode body having a desired shape can be formed. The number of the used mold may be one, or a combination of a plurality of molds may be used. Examples of the case of two molds include two rectangular molds having a rectangular through hole in the center in a plan view (FIG. 9); and a combination of a rectangular mold having a rectangular through hole in the center (FIG. 13A) and a mold that is larger than the rectangular through hole and has two through holes in the center through which the electrode can be inserted (FIG. 13B).

Examples of the gel-forming solution 31 include a solution containing components constituting the gel describe above. The solution may be, for example, an aqueous solution, or an organic solvent solution (e.g. a mixed solution of DMSO and water).

By using a highly viscous solution as the gel-forming solution 31, the gel-forming solution easily penetrates into the holes in the electrodes. Particularly in the case where the conductive base material is a fabric woven from fibers, it is preferable that the gel permeate the conductive base material from the viewpoint of further firmly fixing the electrode to the gel and preventing coming off.

(Gelling Step)

Examples of the method of making the gel-forming solution into a gel with the electrode immersed in the gel-forming solution include a method of repeatedly freezing and thawing, a method of evaporating the solvent of the gel-forming solution, and a method of irradiating the solution with ultraviolet rays.

In the case of the method of repeatedly freezing and thawing, it is preferable to repeat the freezing and thawing at least twice from the viewpoint of further firmly fixing the electrode to the gel and improving the strength of the gel as a substrate.

The freezing may be performed under conditions of a temperature of −30° C. to −15° C. and a time of 120 minutes to 180 minutes, for example.

The thawing may be performed under conditions of a temperature of 0° C. to 25° C. and a time of 20 minutes to 60 minutes.

(Sterilization)

The electrode body of the present embodiment may be used after being sterilized.

The sterilization method is not particularly limited, and examples thereof include high-temperature and high-pressure saturated steam sterilization (autoclave sterilization), gas sterilization, boiling water sterilization, sterilization with a drug (e.g. alcohol, or hypochlorous acid). These sterilization methods can be appropriately used depending on the use of the electrode body.

### EXAMPLES

The following describes the present disclosure in more detail based on Examples. However, the present disclosure is not limited to these Examples.

The following materials were used in Examples.
Polyvinyl alcohol (PVA): Sigma-Aldrich
Dimethyl sulfoxide (DMSO): Wako Pure Chemical Corporation
PDMS: Dow Corning Toray Co., Ltd.
CNT dispersion
Carbon fabric: Toho Tenax
Silicone rubber: AS ONE
Wiring

### Example 1

Electrodes A were prepared with the following method.

### Preparation Method-Electrode A

1) A slide glass was spin-coated with PDMS (PDMS:curing agent=10:1) (1000 rpm, 30 seconds) and heated on a hot plate at 120° C. to cure the PDMS (see FIG. 5A).
2) PDMS (PDMS:curing agent=10:1) was further spin-coated on the cured PDMS (500 rpm, 30 seconds).
3) Carbon fabric was placed on the second spin-coated PDMS before curing the PDMS, and then curing was performed at 120° C. (see FIG. 5B). The carbon fabric used was larger than the layer of PDMS.
4) The carbon fabric and the PDMS were peeled off from the slide glass (see FIG. 5C).
5) The laminate of carbon fabric and PDMS was cut into a ladle shape (see FIG. 5D-1).
6) Fibers frayed in a portion only of carbon fabric on which PDMS was not laminated (FIG. 5D-2), so that one fiber was left and other fibers were removed (see FIG. 5D-3)).
7) Portions other than an exposed portion 24 on one end side and a lead connecting portion 27 on the other end side were covered with PDMS and cured at 120° C. (see FIG. 5E). The dimensions of the obtained electrode A are indicated in FIG. 10. Four types of electrodes A where the lengths a of the leads were 30 mm, 25 mm, 20 mm, and 15 mm were prepared.

The sheet resistance of the conductive base material was 5.2 S)/sq. The sheet resistance is a value measured by the two-terminal method using DL-92 manufactured by KENWOOD.

Subsequently, the electrode A was embedded in a gel.

### Preparation Method-Embedding in Gel

1) Two molds were prepared with silicone rubber. The plan-view shape of the prepared molds is illustrated in FIG. 9.
2) One mold was attached to a slide glass (FIG. 7A), and a 20 wt % PVA solution (a mixture of DMSO and water in a volume ratio of DMSO:water is 4:1 was used as solvent) was poured into the rectangular through hole in the center (FIG. 7B).
3) It was pressed by a slide glass from above, frozen in a freezer at −30° C. for one hour, and then thawed at room temperature for 20 minutes to gel the PVA Solution
4) The upper slide glass was peeled off, another mold of the same shape was stacked with the lead connecting portion on the frame of the mold (FIG. 7C), and a 20 wt % PVA solution same as the above was poured in and pressed from above by a slide glass (FIG. 7D).
5) The PVA solution was gelled by repeating twice a cycle of being frozen in a freezer at −30° C. for one hour and then thawed at room temperature for 20 minutes. Then, the electrode body was taken out from the mold.

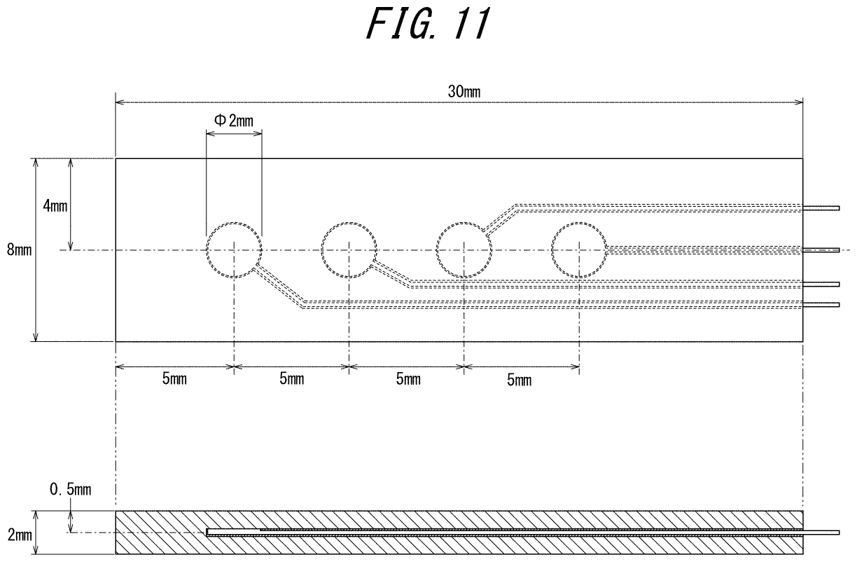

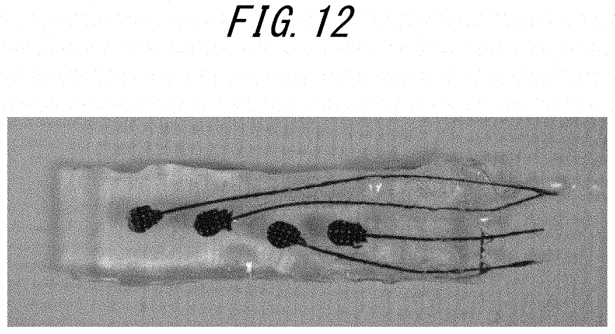

The dimensions of the obtained electrode body are indicated in FIG. 11, and FIG. 12 is a photograph of the obtained electrode body.

### Comparative Example 1

An electrode body was obtained in the same manner as in Example 1 except that a conductive base material having a thickness of 120 μm in which gold was deposited on the surface of a plastic film (product name: Saran Wrap, manufactured by Asahi Kasei Corp.) was used as the conductive base material. The sheet resistance of the conductive base material used was 3.7 S)/sq.

### Example 2

Electrodes B were prepared with the following method.

### Preparation Method-Electrode B

1) A slide glass was spin-coated with PDMS (PDMS:curing agent=10:1) (1000 rpm, 30 seconds) and heated on a hot plate at 120° C. to cure the PDMS (see FIG. 6A).
2) PDMS (PDMS:curing agent=10:1) was further spin-coated on the cured PDMS (500 rpm, 30 seconds).
3) Carbon fabric was placed on the second spin-coated PDMS before curing the PDMS, and then curing was performed at 120° C. (see FIG. 6B).
4) The carbon fabric and the PDMS were peeled off from the slide glass (see FIG. 6C).
5) A few drops of a CNT dispersion were dropped on a contact portion between the carbon fabric and wiring and dried (see FIG. 6D).
6) PDMS was covered on the carbon fabric except for a portion exposed for electrical stimulation, and the PDMS was cured at 120° C. (see FIG. 6D). The dimensions of the obtained electrode are indicated in FIG. 15.

Subsequently, the prepared electrodes were embedded in a gel.

### Preparation Method-Embedding in Gel

1) Two types of molds were prepared with silicone rubber. The plan-view shapes of the prepared molds are illustrated in FIGS. 13A and 13B. Hereinafter, the mold of FIG. 13A is referred to as mold (a), and the mold of FIG. 13B is referred to as mold (b).
2) The mold (a) was attached to a slide glass (FIG. 8A), and a 20 wt % PVA aqueous solution 1 was poured into the rectangular through hole in the center (FIG. 8B).
3) The prepared electrodes were inserted into the mold (b) (FIG. 8C), and the mold (a) was pressed by the mold (b) with the electrodes inserted therein from the above (FIG. 8D).
4) The PVA aqueous solution was gelled by repeating three times a cycle of being frozen in a freezer at −30° C. for one hour and then thawed at room temperature for 20 minutes. Then, the electrode body was taken out from the molds (a) and (b).

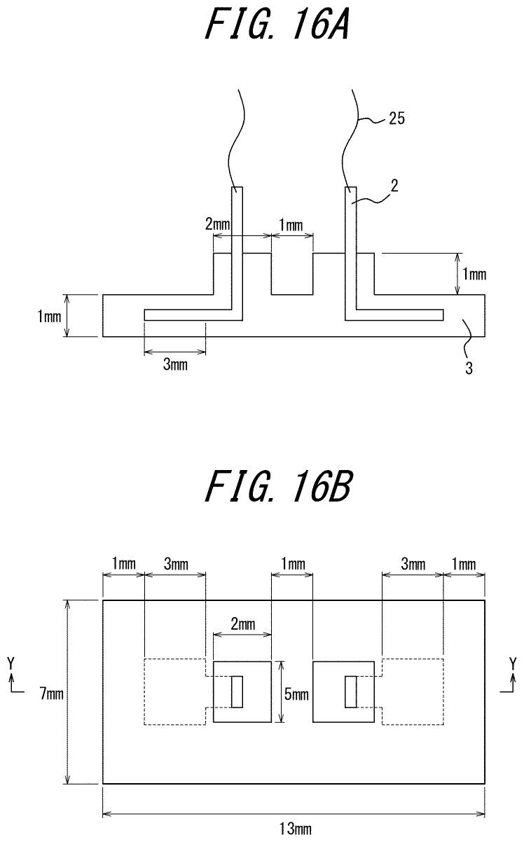

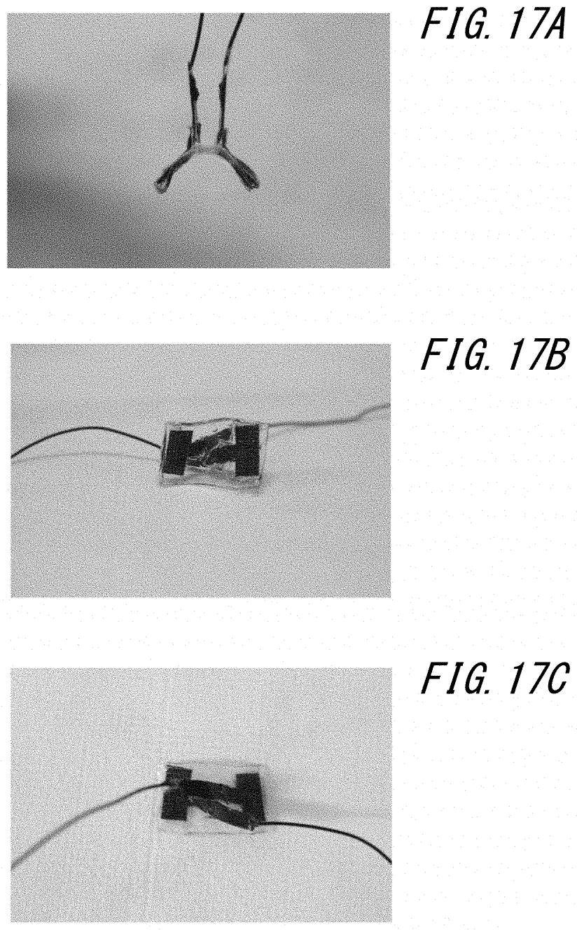

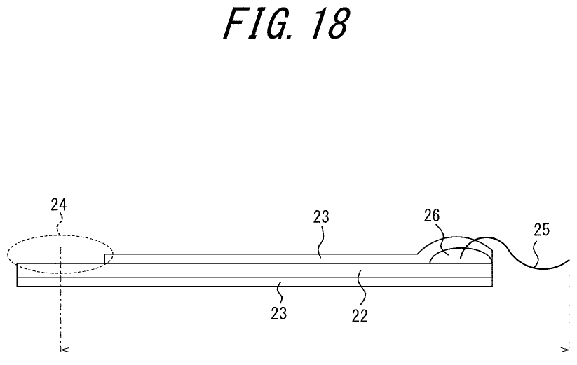

FIG. 14 schematically illustrates the obtained electrode body, and FIGS. 17A to 17C are photographs of the obtained electrode body. FIG. 17A is a photograph taken from the side, FIG. 17B is a photograph taken from the back (electrode exposed portion), and FIG. 17C is a photograph taken from the front (taken-out wiring portion). The dimensions of the electrode body are indicated in FIGS. 16A and 16B. Note that the size of this Example is the size of the case where a rat is used as an experimental animal.

The sheet resistance of the carbon fabric used was 5.2 S)/sq.

The resistance from the tip of the wiring of the prepared electrode to the center of the exposed portion of the carbon fabric (see FIG. 18) was measured by the two-terminal method using DL-92 manufactured by KENWOOD, and it was 30Ω to 60Ω.

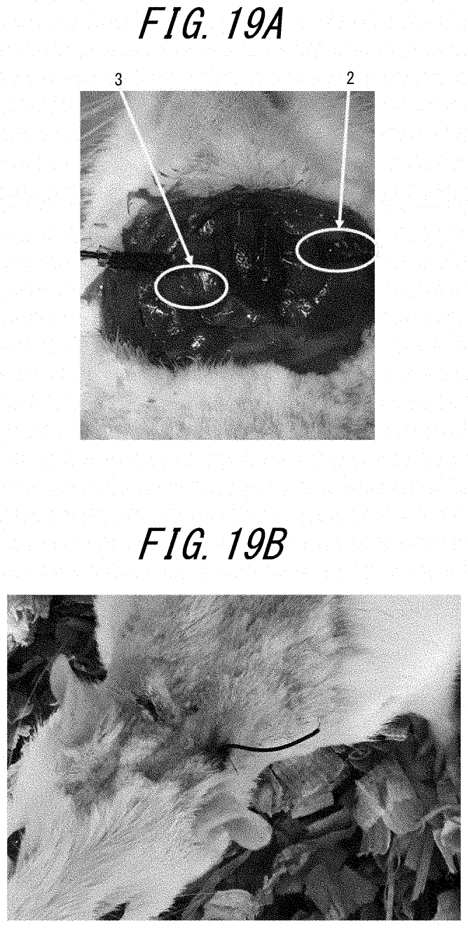

The electrode body prepared in Example 1 was embedded behind the neck of a rat. FIG. 19A is a photograph of the state of being embedded in a rat, and FIG. 19B is a photograph of the wiring taken out from the back of the neck after the operation.

(Evaluation)

### (Followability to Gel)

A carbon fabric similar to that of Example 1 was cut into a size of a length of 10 mm, a width of 50 mm, and a thickness of 300 μm, and covered with a 20 wt % PVA solution (a mixture where a volume ratio of solvent:DMSO to water was 4:1) to obtain a sample (CF 300 μm) having a length of 30 mm, a width of 30 mm, and a thickness of 1000 μm. The conditions of gelling of PVA were the same as in Example 2.

A sample (CF 600 μm) was obtained in the same manner as the sample (CF 300 μm) except that two carbon fabrics were stacked. A sample (CF 900 μm) was obtained in the same manner as the sample (CF 300 μm) except that three carbon fabrics were stacked. The flexibility was evaluated using the sample (CF 600 μm) and the sample (CF 900 μm).

The three types of samples were placed on a thin glass rod and left at a temperature of 25° C. for one minute.

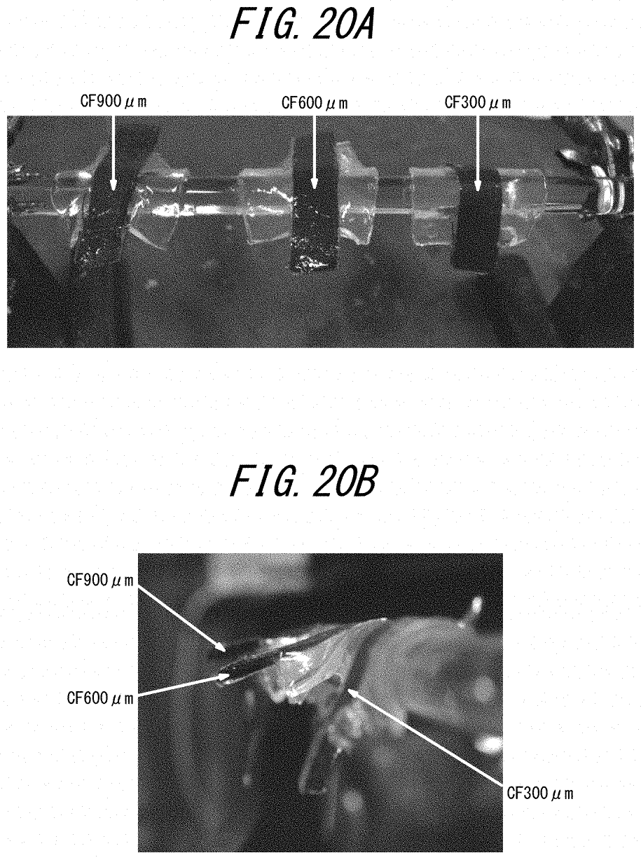

The results are illustrated in FIGS. 20A and 20B. FIG. 20A is a view taken from the above of the samples after the test, and FIG. 20B is a view taken from the side. The sample (CF 300 μm) followed the flexible movement of the PVA gel, and deformation of the PVA gel was hardly observed. Therefore, the sample (CF 300 μm) had excellent followability to the gel. On the other hand, in the cases of the sample (CF 600 μm) and the sample (CF 900 μm), the PVA was largely deformed, and the followability to the gel was poor.

(Electric Double Layer Capacity)

The electrode bodies prepared in Example 1 and Comparative Example 1 were subjected to cyclic voltammetry measurement and impedance measurement using an electrochemical measurement system (product number: Model 760C, manufactured by CH Instruments, Inc.). An Ag/AgCl electrode was used as a reference electrode, and the electrode at the exposed portion was used as a working electrode.

In the cyclic voltammetry measurement, the sweep potential was 0 V to 0.5 V, and the sweep rate was 0.05 mV/s.

In the impedance measurement, the two-terminal method was used to apply an AC voltage having a frequency of 1 Hz to 100000 Hz and an amplitude of 0.05 V to the electrodes to perform the measurement.

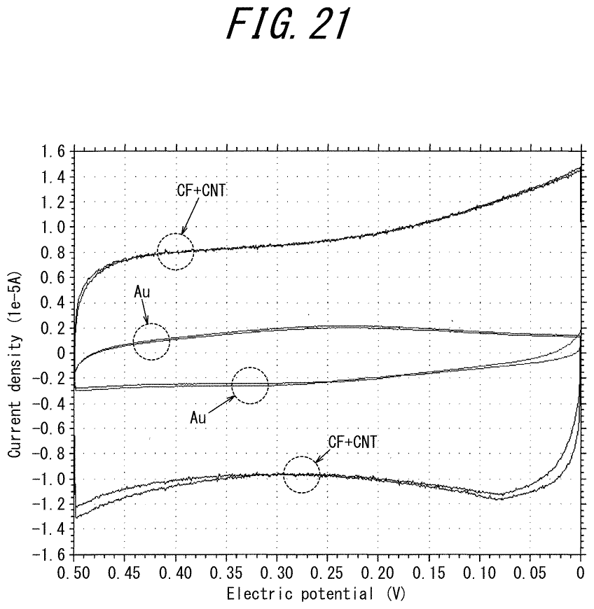

The electric double layer capacity was 1.7×10⁻⁴ F/cm² for gold and 8.5×10⁻⁴ F/cm² for carbon fabric (FIG. 21). The electrode body of Example 1 had a larger electric double layer capacity than the electrode body of Comparative Example 1, which was about 5 times. It is assumed that the electrode body of Example 1 has a large electric double layer capacity and is unlikely to undergo electrolysis.

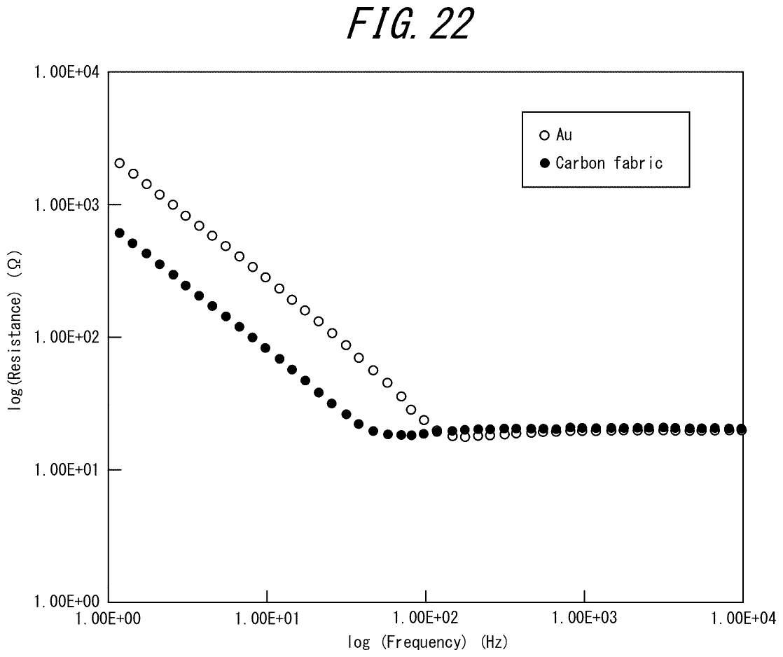

In addition, the impedance of Example 1 was lower than that of Comparative Example 1 (FIG. 22).

(Combination of Gel and Electrode)

A carbon fabric similar to that of Example 1 was cut into a size of a length of 10 mm, a width of 10 mm, and a thickness of 300 μm, and covered with a 20 wt % PVA solution (a mixture where a volume ratio of solvent:DMSO to water was 4:1) to obtain a sample (CF) having a length of 30 mm, a width of 30 mm, and a thickness of 1000 μm. The conditions of gelling of PVA were the same as in Example 2.

An electrode in which gold was deposited on the surface of a plastic film with a smooth surface (product name: Saran Wrap, manufactured by Asahi Kasei Corp.) was covered with a 20 wt % PVA solution (a mixture where a volume ratio of solvent:DMSO to water was 4:1) to obtain a sample (plastic film) having a length of 10 mm, a width of 10 mm, and a thickness of 120 μm. The conditions of gelling of PVA were the same as in Example 2.

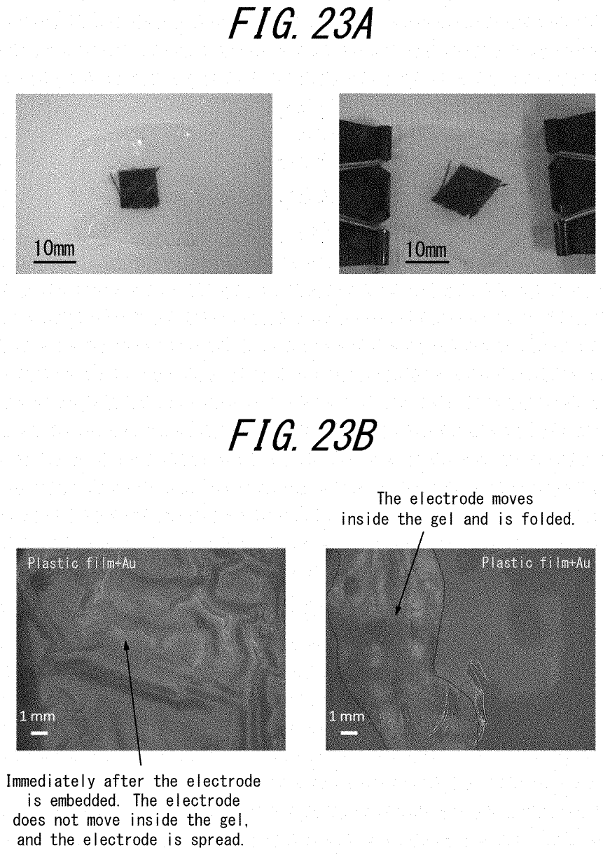

The samples were being pulled in the width direction (the cross direction in FIGS. 23A and 23B) for one minute. The sample (CF) did not move in the PVA gel and was firmly fixed to the PVA gel (FIG. 23A). On the other hand, the sample (plastic film) moved greatly in the PVA gel and was inferior in fixability (FIG. 23B).

(Mechanical Strength)

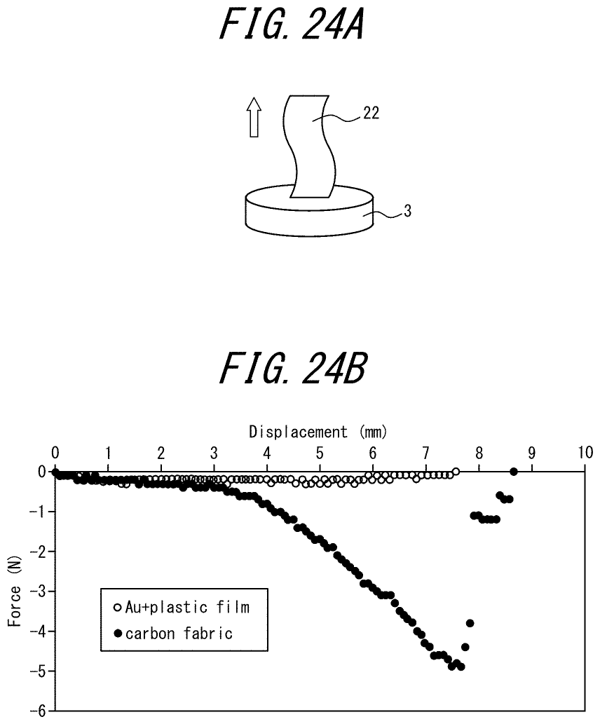

A carbon fabric similar to that of Example 1 was cut into a size of a length of 5 cm, a width of 1 cm, and a thickness of 300 μm, and a portion of 1 cm from one end in the length direction was covered with a 20 wt % PVA solution (a mixture where a volume ratio of solvent:DMSO to water was 4:1) to obtain a test piece (CF) (FIG. 24A). The conditions of gelling of PVA were the same as in Example 2.

A conductive base material in which gold was deposited on a plastic film similar to that of Comparative Example 1 was cut into a size of a length of 5 cm, a width of 1 cm, and a thickness of 120 μm, and a portion of 1 cm from one end in the length direction was covered with a 20 wt % PVA solution (a mixture where a volume ratio of solvent:DMSO to water was 4:1) to obtain a test piece (plastic gold-deposited film) (FIG. 24A). The conditions of gelling of PVA were the same as in Example 2.

The other end of the test piece, which was not covered with the PVA gel, was pulled upward in FIG. 24A.

The results are illustrated in FIG. 24B.

As the force gradually increased, the carbon fabric of the test piece (CF) (carbon fabric) gradually expanded, and the carbon fabric was torn when the force reached 4.9 N. During the test, the carbon fabric and the PVA gel were firmly fixed together.

On the other hand, for the test piece (plastic gold-deposited film) (Au+plastic film), the plastic gold-deposited film came off from the PVA gel when the force reached 0.1 N.

### Example 3

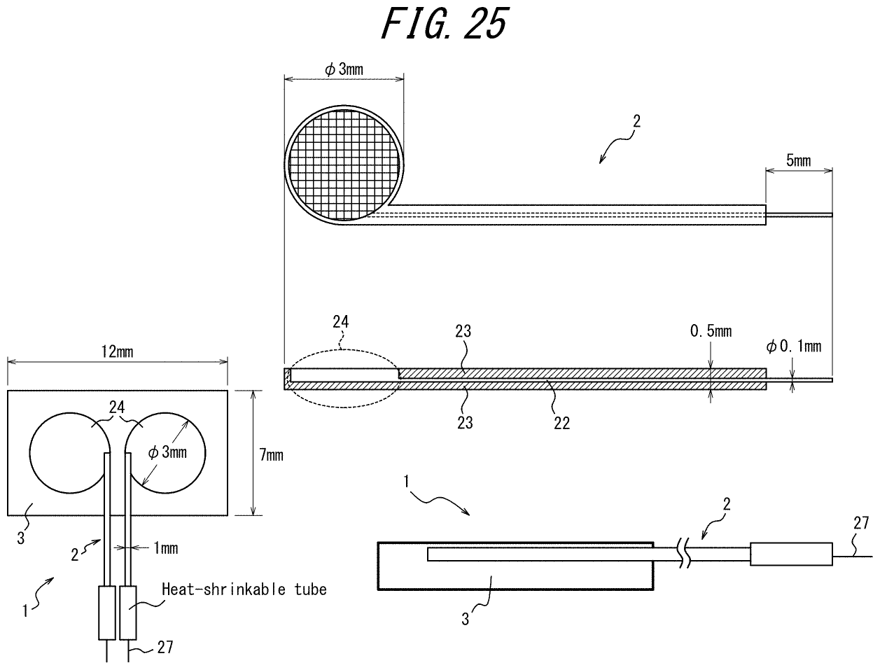

Electrodes C were prepared with the following method.
1) A slide glass was spin-coated with PDMS (PDMS:curing agent=10:1) (1000 rpm, 30 seconds) and heated on a hot plate at 120° C. to cure the PDMS (see FIG. 6A).
2) PDMS (PDMS:curing agent=10:1) was further spin-coated on the cured PDMS (500 rpm, 30 seconds).
3) Carbon fabric was placed on the second spin-coated PDMS before curing the PDMS, and then curing was performed at 120° C. (see FIG. 6B).
4) The carbon fabric and the PDMS were peeled off from the slide glass (see FIG. 6C).
5) The laminate of carbon fabric and PDMS was cut into a ladle-like shape (see FIG. 25). Note that a portion (φ 0.1 mm, lead connecting portion) only of carbon fabric on which PDMS was not laminated was left at one end (see FIG. 25).
6) The vicinity of the boundary between the laminate of carbon fabric and PDMS and the lead connecting portion was covered with a heat-shrinkable tube and subjected to thermocompression bonding.
7) Portions where the carbon fabric and the PDMS were laminated were covered with PDMS except for a portion of carbon fabric exposed for electrical stimulation, and curing was performed at 120° C. (see FIG. 25).
8) Poly (3,4-ethylenedioxythiophene) (PEDOT), which was extremely minute compared to the uneven surface of the carbon fabric, was subjected to electrolytic polymerization so as to form particles on the carbon fabric portion exposed for electrical stimulation so that the uneven surface remained exposed. It was washed and then dried.

### Preparation Method-Embedding in Gel

Subsequently, the prepared electrode was embedded in a gel.
1) A mold (c) having a plan-view shape as in FIG. 13A was prepared with silicone rubber.
2) The mold (c) was attached to a slide glass (FIG. 8A), two electrodes were arranged in the center of the rectangular through hole in the center, and then a 20 wt % PVA aqueous solution 1 was poured in (FIG. 8B). The electrodes were slightly submerged in the 20 wt % PVA aqueous solution.
3) The PVA aqueous solution was gelled by repeating three times a cycle of being frozen in a freezer at −30° C. for 10 minutes and then thawed in a refrigerator for 10 minutes. Then, the electrode body was taken out from the mold (c) to obtain a hydrogel organic electrode (see FIG. 25).

The shape and dimensions of the obtained hydrogel organic electrode are illustrated in FIG. 25.

### (Electroencephalogram Measurement)

All the animal experiments were conducted with the approval of Animal Health Committee of Tohoku University Graduate School of Medicine.

An adult rat was continuously anesthetized during the period of craniotomy and neurography. The head of the anesthetized rat was fixed to a fixing device, and a cortical region of 12 mm×12 mm of both hemispheres were exposed by craniotomy. The rat and the fixing device were placed inside a Faraday cage with a metal wire base to reduce noise.

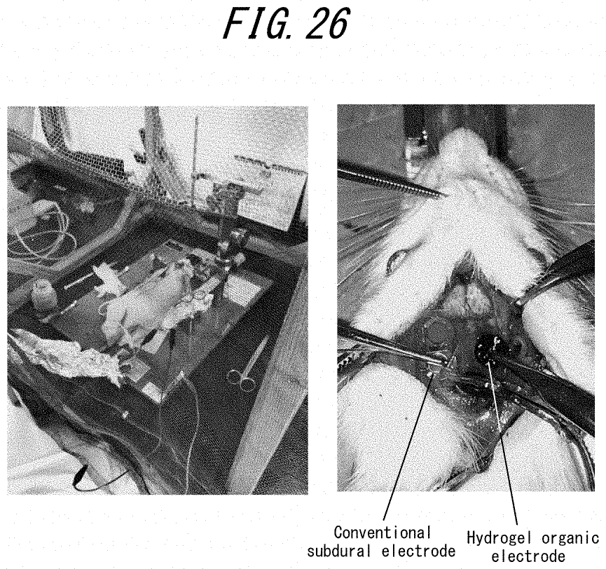

A conventional subdural electrode (manufactured by Unique Medical, a conventional product that was an intracranial electrode and an electrode in which a flat metal was covered with a resin, and that did not meet the requirements of claim 1 of the present application) and the prepared hydrogel organic electrode (electrode body) were placed on the exposed cortex of the rat and fixed with tweezers (FIG. 26). All recordings were made with reference to the electrodes attached to the body of the rat. Neural data were acquired by an amplifier (FE135 Dual Bio Amp, ADInstruments) and a data acquisition system (PowerLab 8/35, ADInstruments). Neurographic data were analyzed offline using LabChart v8 software (ADInstruments). The power spectrum of the recorded data was analyzed by fast Fourier transform. The signal-to-noise ratio (S/N ratio) was calculated from the data with S/N[dB]=10 log₁₀ (μ²/σ²), where μ: average, and σ: variance.

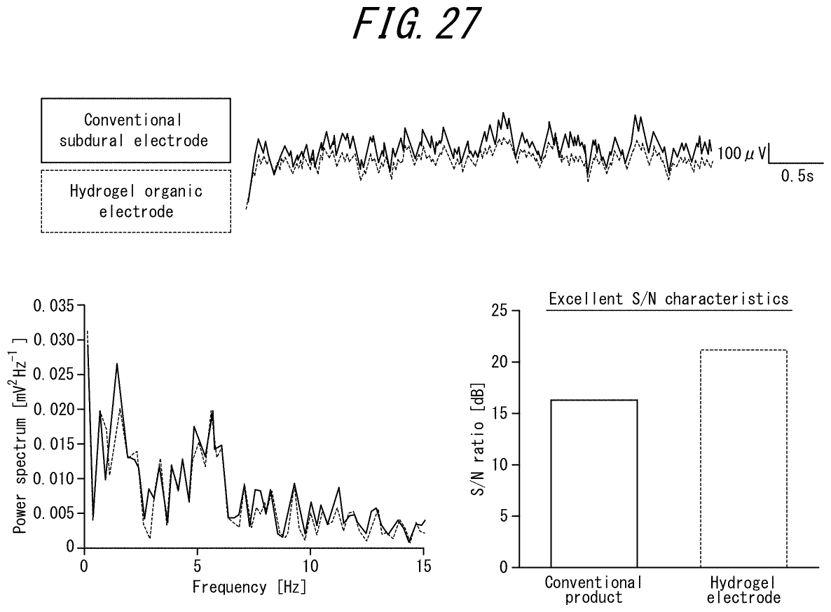

The potential waveform was successfully measured with the conventional subdural electrode and the hydrogel organic electrode (FIG. 27 upper right). The waveform and amplitude of the recorded data were similar to rat electroencephalogram measured in other literature, suggesting that electroencephalogram was successfully obtained with the hydrogel organic electrode and the conventional subdural electrode. The power spectrum of the neural data also suggests that 0 Hz to 15 Hz brain waves were recorded by both the conventional hydrogel electrode and the hydrogel organic electrode (FIG. 27 lower left). High frequency noise convolved on the electroencephalogram was more frequently seen in the waveform measured by the conventional subdural electrode than that of the hydrogel organic electrode. The signal-to-noise ratio (S/N ratio) of the recorded data suggests that the hydrogel organic electrode has better S/N characteristics than the conventional subdural electrode (FIG. 27 lower right). The high S/N ratio is probably a result of the low electrical impedance of the hydrogel organic electrode and the high adhesion of the hydrogel to the rat brain because of its flexibility.

### INDUSTRIAL APPLICABILITY

In the electrode body of the present disclosure, the electrode is firmly fixed to the gel. Therefore, the electrode body of the present disclosure can be used as a gel electrode or the like to be embedded in a living body.

### REFERENCE SIGNS LIST

1 electrode body
2 electrode
21 uneven surface
22 conductive base material
23 resin layer
24 exposed portion
25 wiring
26 connecting portion
27 lead connecting portion
28 curve
29 substrate for forming electrode
3 gel
31 gel-forming solution
4 electrode body mold
5 substrate for forming electrode body

1. An electrode body, wherein
at least a part of a surface of an electrode is uneven, and at least a part of the uneven surface of the electrode is covered with a gel,
the gel has a three-dimensional network structure,
the at least a part of the uneven surface is in contact with the three-dimensional network structure and is embedded inside the three-dimensional network structure,
the electrode comprises a conductive base material having a network structure on a surface and inside, the three-dimensional network structure of the gel enters the network structure, the network structure and the three-dimensional network structure are entangled with each other inside the conductive base material, and the three-dimensional network structure passes through the inside of the conductive base material and penetrates in a thickness direction of the conductive base material, and
the electrode is a laminated body in which an insulating resin layer is laminated on both surfaces of the conductive base material.

2. The electrode body according to claim 1, wherein the electrode has a thickness of 1 μm to 500 μm.

3. (canceled)

4. The electrode body according to claim 1, wherein the conductive base material is a fabric woven from fibers, and the uneven surface is a surface of the fabric.

5. The electrode body according to claim 4, wherein the conductive base material is carbon fabric.

6. The electrode body according to claim 1, wherein at least a part of the surface of the electrode comprises an exposed portion where the conductive base material is exposed.

7. The electrode body according to claim 6, wherein the exposed portion is in contact with the three-dimensional network structure and is embedded inside the three-dimensional network structure.

8. The electrode body according to claim 1, wherein the gel is a hydrogel.

9. The electrode body according to claim 1, wherein the gel comprises polyvinyl alcohol.

10. The electrode body according to claim 1, wherein the electrode is curved at at least one location, and the curve is in contact with the three-dimensional network structure and is embedded inside the three-dimensional network structure.

11. The electrode body according to claim 1, comprising two or more of the electrodes.

12. A method of producing an electrode body, which is a method of producing the electrode body according to claim 1, comprising
an electrode forming step of forming an electrode,
an immersion step of immersing the electrode in a gel-forming solution, and
a gelling step of making the gel-forming solution into a gel with the electrode immersed in the gel-forming solution.

13. The method of producing an electrode body according to claim 12, wherein freezing and thawing are repeated twice or more in the gelling step.

14. (canceled)

15. The method of producing an electrode body according to claim 12, wherein the electrode has a thickness of 1 μm to 500 μm.

16. (canceled)

17. The method of producing an electrode body according to claim 12, wherein the conductive base material is carbon fabric.

18. The method of producing an electrode body according to claim 12, wherein at least a part of a surface of the electrode comprises an exposed portion where the conductive base material is exposed.

19. The method of producing an electrode body according claim 12, wherein the gel is a hydrogel.

20. The method of producing an electrode body according to claim 12, wherein the gel comprises polyvinyl alcohol.

21. The electrode body according to claim 1, which is used by directly contacting the gel with a living body.
